(19) United States
(12) Patent Application Publication
(10) Pub. No.: US 2005/0131489 A1
(43) Pub. Date: Jun. 16, 2005

(54) Therapeutic device for electrical muscle stimulation treatment of venous deficiency functional disorders of the lower limbs and for preventing deep venous thrombosis

(76) Inventor: Christian Gardon-Mollard, Chamalieres (FR)

Correspondence Address: JACOBSON HOLMAN PLLC, 400 SEVENTH STREET N.W., SUITE 600, WASHINGTON, DC, 20004, US

(21) Appl. No.: 10/498,323

(22) PCT Filed: Dec. 17, 2002
(86) PCT No.: PCT/FR02/04408

(30) Foreign Application Priority Data
Dec. 17, 2001 (FR) 01/16294

(51) Int. Cl. A61N 1/18

(52) U.S. Cl. 607/49

(57) ABSTRACT

The device (10) comprises a knitted article of the stocking, sock, or tights type having a leg portion suitable for coming into contact with the skin of the lower limb. At least in a portion of its surface in contact with the lower limb, the device comprises a knitted zone (24) that is electrically conductive and that is connected to one terminal of an electromyostimulation generator (12). The height of the conductive zone (24) is defined to cover a extent of a selected region of the lower limb, in particular the calf, thigh, or foot region, and the stitch of the knit in this conductive zone includes a filler yarn and/or a face yarn that is electrically conductive. Advantageously, the knit and the yarn of the article are selected in such a manner as to form a compressive orthosis suitable for delivering therapeutic compressive support that decreases from the ankle.

The present invention relates to treating functional disorders of venous insufficiency in the lower limbs, and to preventing deep venous thromboses.

A strong medical consensus accepts that degressive medical elastic supportive hose associated with walking are beneficial in respect of functional manifestations of venous insufficiency in the lower limbs, and the beneficial effects of contracting the muscles of the calf by putting into action the so-called “calf muscle pump” have been known for a long time, said pump significantly stimulating venous return from the lower limbs.

This recommendation cannot be followed by people who are very old or crippled, or by people placed in particular situations of immobility (a typical case being that of long-haul air travel), where a state of total or partial immobilization increases the risks associated with venous stasis.

A comparable situation often occurs in treating leg ulcers with patients who are often not keen to take exercise, and more generally in preoperative, peroperative, and postoperative situations with patients who are bedridden, under anesthetic, or in intensive care.

One of the objects of the invention is to propose a therapeutic appliance for treating the functional disorders of venous insufficiency in the lower limbs in the situations mentioned above, making it possible to remedy the various manifestations in the lower limbs that are associated with venous stasis, and generally, with all situations of exaggerated venous stasis caused by lengthy immobility, and in particular for preventing deep venous thrombosis (DVT).

To this end, the invention proposes implementing a device comprising a knitted article of the stocking, sock, or tights type with a leg portion suitable for coming into contact with the skin of the lower limb, characterized in that it comprises, at least in a portion of its surface in contact with the lower limb, a knitted zone that is electrically conductive, in that the height of the conductive zone is defined in such a manner as to cover the extent of a selected region of the lower limb, in particular the calf and/or thigh and/or foot region, and in that the stitch of the knit in this conductive zone includes a filler yarn and/or a face yarn that is electrically conductive. The area of the conductive zone is typically not less than 100 square centimeters (cm²), and is preferably at least 400 cm².

The invention also provides, as such, a therapeutic appliance comprising, in combination, an electromyostimulation generator suitable for delivering pulses of current between its output terminals; a device as set out above; and a connection wire electrically connecting the electrically conductive region of said device to a respective terminal of the electromyostimulation generator.

Proposals have indeed been made to use electrical stimulation appliances for causing passive muscle contraction by localized stimulation of certain points on the skin; however those devices require adhesive or carbon electrodes to be put into place on the skin (where carbon electrodes need to be moistened), thus requiring complicated handling for suitably placing the electrodes, fixing them, putting them back into place if they become detached from the skin, etc.

WO-A-01/02052 describes a garment locally incorporating in the cloth conductive zones for constituting electrodes so as to make it easier to position said electrodes in defined locations and to hold them in place, a considerable constraint given the relatively small size of the electrodes and thus the need to place them at well-determined points on the body in order to obtain the looked-for effect, for example picking up electrographic signals, transcutaneous stimulation of certain nerves, etc.

In contrast, the invention makes it possible to implement muscle excitation on request without putting any electrode into place, merely by means of the fabric, and to do so in diffuse manner over the entire extent of the electrically conductive zone of the knitted article.

Most advantageously, the stitch and the yarn of the article are selected in such a manner to form a compressive orthosis suitable for delivering compression that is degressive from the ankle, the compression being therapeutic, i.e. with pressure at the ankle of at least 10 millimeters of mercury (mmHg) (13.3 hectopascals (hPa)).

It is thus possible to combine the effects of electrical stimulation with the effects of the compressive support provided by the stocking, thus making it possible in particular to benefit from the highly advantageous venous return effect without the drawbacks of excessive compressive support (for this purpose, it is possible to select a stocking in a low or medium support class, e.g. classes I or II). In addition, even when the electrical stimulation is stopped, the patient can continue to benefit from the advantages and the comfort of light compressive support.

This characteristic can be advantageous, for example for people in situations of lengthy immobilization, such as while traveling or car or by air.

This characteristic is also advantageous in that for a given orthosis and a given patient, it enables a “two-stage” effect to be obtained on venous circulation: a first effect is permanent and is the result of the compressive support provided by the stocking, and where necessary there is an additional effect that is adjustable in duration and in intensity obtained by putting the electrical stimulation into operation, e.g. for a patient who is bedridden, immobilized, or in a coma.

The article of the invention may include an upper electrically conductive region separated from said conductive zone by an intermediate region that is not conductive, said intermediate region having an electrical connection passing therethrough connecting the upper region to the conductive zone, such that the upper region forms a connection strip or area for a wire for connection to a terminal of an electromyostimulation generator. The electrical connection is advantageously a narrow knitted strip of the article that includes an electrically conductive knit yarn and/or filler yarn.

The conductive yarn is in particular a yarn including a thread of conductive material, e.g. a material from the group comprising silver, gold, copper, and copper oxide. The thread of conductive material is advantageously a covering thread, in particular for parallel covering.

Preferably, the face of the conductive zone that is to come into contact with the skin of the lower limb is that one of the faces which presents the largest apparent area of the conductive thread.

There follows a description of an embodiment of the invention, given with reference to the accompanying drawings.

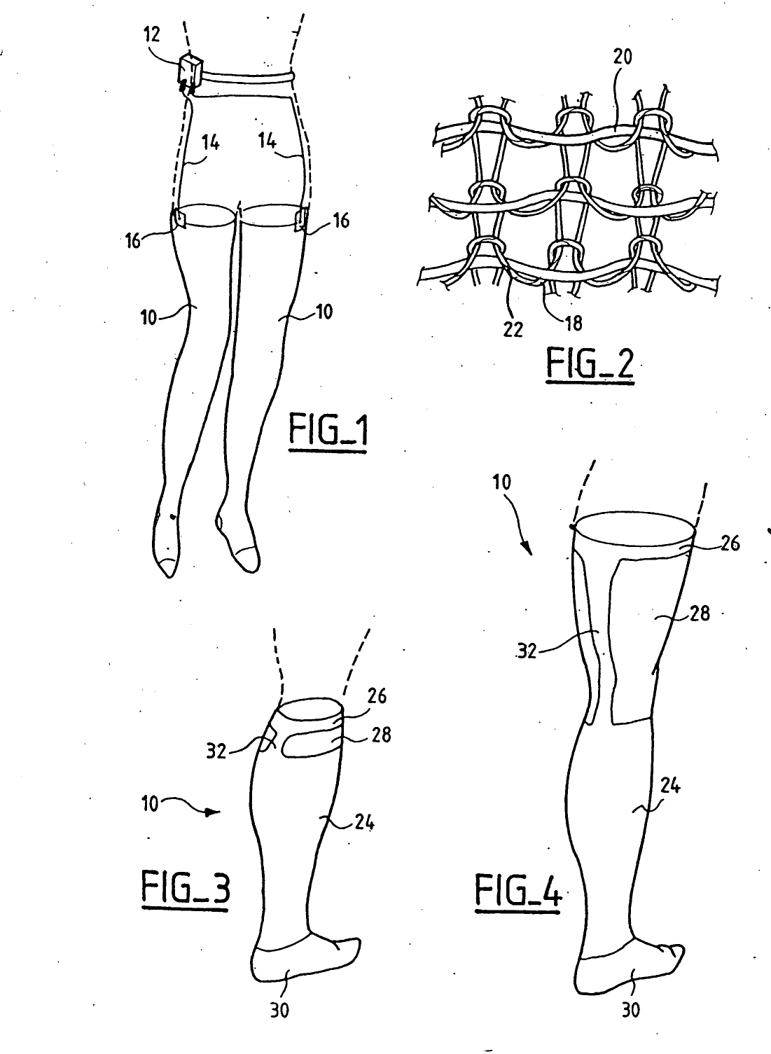

FIG. 1 is a general view showing the various elements forming the therapeutic appliance of the invention.

FIG. 2 shows in detail the knitted stitching of the article used by the appliance of the invention.

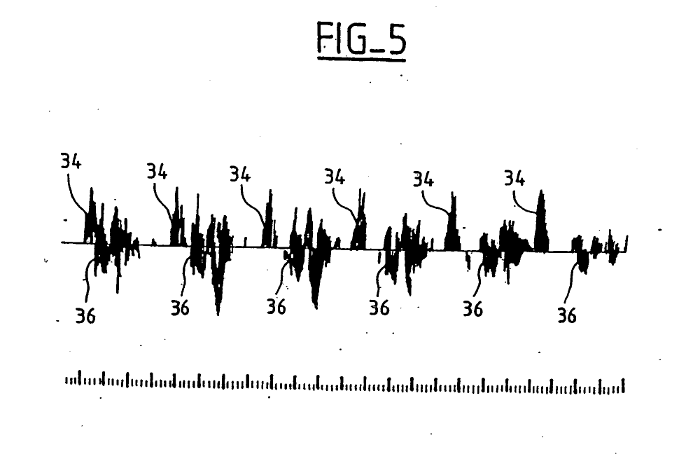

FIGS. 3 and 4 show two possible variants for the knitted article.

FIG. 5 is a Doppler echograph recording corresponding to the blood flow obtained when using the appliance of the invention.

In FIG. 1, reference 10 designates a stocking of the invention having incorporated therein, in a manner that is described in greater detail below, a conductive metal thread occupying all or part of the stocking so as to be capable of stimulating muscles directly by means of the fabric. Although FIG. 1 shows an article of the thigh-length type, the invention is applicable to all models of surgical stocking such as socks, thigh-length stocking, tights, tubular compressive orthoses (in the form of a sleeve not having a foot or a heel portion), etc.

Each stocking is connected to one terminal of an electrostimulation generator 12, e.g. worn at the belt, by means of connection conductors 14 connected to respective conductive regions of the stocking 10 by any appropriate means, e.g. a “crocodile” or “alligator” type clip 16, or the like.

For the electrostimulation generator, it is possible to use a commercially available model of known type, replacing the electrodes of the appliance with the stockings 10 of the invention, connected to the generator via respective connection conductors 14.

By way of example, a suitable appliance is the generator of the Microstim INSEP One device (manufactured by Valmed SA, Switzerland) initially intended for electromyostimulation, which delivers current at low frequency (in the range 1.25 hertz (Hz) to 1.75 Hz) in the form of asymmetrical two-phase pulses and with zero net current. In open circuit, the peak voltage of the pulses is 45 volts (V)±10%. With a load of 500 ohms (Ω), the voltage is less than 5 volts root mean square (rms)±10% for a peak current of 88 milliamps (mA)±10%.

There follows a description of how all or part of the stocking 10 is made to be conductive.

This characteristic is achieved most advantageously by incorporating an optionally metal conductive thread in the knitting yarn, e.g. in the form of a first or second covering thread, it being understood that this thread must come into direct contact with the skin. It may thus be either a single covering, or else a second covering when there are two coverings. The covering is preferably parallel covering, e.g. made by using “air jet” mixing and texturing technology, instead of a more traditional rotary covering.

The covered conductive yarn can be used either as the knit yarn or as a filler yarn, or even for both kinds of yarn, depending on the skin contact area that is desired for a given model.

FIG. 2 shows an example of knitting implemented in this manner with a knit yarn 18 and a filler yarn or a face yarn 20. In the example shown, the conductive yarn 22 is a polyamide yarn covered in a surface silver layer, e.g. a yarn of the (trademark) type produced by Nobel Fiber Technologies, Scranton, Pa., USA. This yarn contains about 15% pure silver mixed with the polyamide fiber. It is itself known, but in the medical or hygiene field it has previously been proposed only for the following properties:

antimicrobial properties (a property that is traditionally recognized as belonging to silver and its salts);

anti-odor properties, due to the catalytic effect of silver, in particular with respect to ammonia and denatured proteins; and

antistress properties: the conductive fabric is believed to pick up and dissipate triboelectric charge accumulated on the surface of the skin.

In the invention, this yarn is used for its metallic conductivity properties, so as to form a conductive surface of large area in direct contact with the skin, thus enabling electricity to be conducted and diffused through the skin by the natural moisture of the skin (and/or by spraying an aqueous mist, e.g. by means of a commercially available mineral water spray can, so as to increase the moisture content of the fabric).

The choice of silver as the conductive material is not limiting, and other conductive materials could be envisaged, in particular metals based on copper and copper oxide, or indeed non-metallic materials such as filled polymers.

The face of the conductive zone that is to come into contact with the skin of the legs is advantageously that one of the faces that presents the largest apparent surface area of conductor yarn. To enable stimulation to be diffuse, the area over which the conductive zone 24 extends should be not less than 100 cm², and preferably at least 400 cm².

The knit yarn, and above all the filler yarn, may be selected so as to produce a small amount of therapeutic compressive support, typically in class I or II (classification in accordance with French standards) with pressure at the ankle being in the range 10 mmHg to 15 mmHg (13.3 hPa to 20 hPa) for class I and in the range 15 mmHg to 20 mmHg (20.0 hPa to 26.6 hPa) for class II. To obtain a pressure gradient that tapers off regularly from the ankle to the thigh, in order to facilitate venous return, the support pressure decreases from the ankle to the thigh, with pressure at the top being 50% less than pressure at ankle level.

Such medical compressive support is itself well known, however in the context of the present invention, it is advantageously be combined with electrical stimulation, thus making it possible to select a lower support class, thereby obtaining more positive effects on venous return without the drawbacks of a high degree of compressive support.

FIGS. 3 and 4 show two ways in which the conductive zone of the stocking 10 can be configured. FIG. 3 shows an example of a knee-length stocking covering the calf, and FIG. 4 shows an example of a thigh-length stocking, going higher up but likewise intended for stimulating the muscles of the calf.

In these two figures, reference 24 designates the electrically conductive zone that receives and diffuses into the skin the electrical current produced by the electrostimulation generator. This zone extends substantially from the ankle to just below the knee, so as to stimulate the calf muscles. To enable the clip 16 which is connected to the electrostimulation generator to be connected, the top edge 26 which is separated from the conductive zone 24 by a non-conductive zone 28 is likewise made to be conductive: it then suffices to attach a small crocodile clip connected to the generator to this edge 26 in order to connect the appliance electrically.

Like the intermediate zone 28, the bottom zone 30 covering the foot is non-conductive. It may even be omitted, in which case the article is no longer in the form of a sock or stocking, but rather in the form of an open sleeve not having any foot or heel.

The electrical connection between the conductive zone 24 and the top zone 26 to which electricity is supplied may be achieved in various ways:

either by a specific electrical conductor that is not included in the knit;

or else, and preferably, by a knitted strip 32 of a certain width that incorporates in its structure the conductive yarn (knit yarn and/or filler yarn) without any interruption between the zones 24 and 26.

Compared with the first solution, the second solution presents the advantage of being tough, insofar as a break in a conductor yarn at certain locations, e.g. during washing or handling, will have no practical incidence on electrical continuity, unlike a single wire or a linear conductor (much more fragile).

The results of a preliminary clinical study of the hemodynamic effects of stimulating calf muscles using an appliance of the invention are given below.

The purpose of the study was to observe and quantify the effects of intermittent electrical stimulation on the “calf muscle pump” in terms of return venous blood flow in healthy subjects at rest.

It is known that the physiological muscle contractions that occur in the standing position, while walking, or running, lead to venous blood being expelled centripetally, essentially over the deep and muscular venous networks. The study sought to observe the magnitude of the expulsion of deep venous blood in an immobile subject, by provoking involuntary muscular contraction by electrical stimulation.

The study was performed on a healthy male volunteer aged 19 (183 cm, 85 kilograms (kg)) who presented no past history of venous disease, or of old or recent complications of venous disease.

Waves of deep venous blood flow were observed by means of a Doppler echograph appliance using a three-frequency annular sector probe set at 7 megahertz (MHz) for observing the deep veins of the leg, and a continuous Doppler probe at 10 MHz for making recordings.

Flow was measured in the popliteal vein in the sitting position.

Before the study, a Doppler echograph examination of the superficial and deep veins of the lower limbs of the subject was undertaken; it was found to be normal, in particular concerning the valve function of the entire venous network of the legs, (i.e. both the superficial network and the deep network).

The patient then put on a conductive stocking sleeve which was placed on the soleus muscles of both legs.

Each end of the conduction cable of the generator was placed under the sleeve in contact with the skin, beneath the top edge of the sleeve (after the terminal electrodes had been removed).

The patient himself manually increased the pulses up to the bearable pain threshold, i.e. notch 39 on the dial of the generator (which goes up to 40), leading to visible muscular contraction.

Observation showed involuntary muscular contraction of the calf muscles that was clearly visible, with the intensity of the contraction being proportional to the magnitude of the pulses as given on the dial of the generator. Muscular contraction took place not only under the connection zone for the connection wires of the generator, but also at a distance therefrom, revealing good electrical conduction of the fabric. The volunteer said he could feel the pulse in the calf and up to the thigh.

The Doppler and echographic observations of the blood flows in the right popliteal vein while the patient was in the sitting position reveal (recording reproduced in FIG. 5):

at 34 in FIG. 5: regular arterial flow of cardiac origin, represented by peaks at a regular frequency; and

at 36 in FIG. 5, provoked venous flow, repeated on each involuntary muscle contraction, revealing that the appliance of the invention created an artificial venous pulse, thereby achieving genuine “arterialization” of the vein.

Observation also showed the magnitude of the blood flow is optimized for the sitting position, i.e. when the muscles involved for maintaining the standing position are at rest.

In conclusion, the study confirms the good electrical conduction of the fabric of the stocking, and also the quality of the muscular contraction which, physiologically, increases the return blood flow.

The study confirms that with an immobile subject whose muscles are not functioning or are no longer functioning, it is possible to enhance venous return in the absence of ambulation, thereby inducing an antistasis effect, and additionally associating degressive compressive support due to the therapeutic effect of the surgical stocking.

1: A device (10) for treating functional disorders of venous insufficiency in the lower limbs and for preventing deep vein thromboses,
the device being of the type comprising a knitted article of the stocking, sock, or tights type with a leg portion suitable for coming into contact with the skin of the lower limb,
the device being characterized in that it comprises, at least in a portion of its surface in contact with the lower limb, a knitted zone (24) that is electrically conductive,
in that the height of the conductive zone (24) is defined in such a manner as to cover the extent of a selected region of the lower limb, in particular the calf and/or thigh and/or foot region, and
in that the stitch of the knit in this conductive zone includes a filler yarn (18) and/or a face yarn (20) that is electrically conductive.

2: The therapeutic knitted article of claim 1, in which the area of said conductive zone (24) is not less than 100 cm².

3: The therapeutic knitted article of claim 2, in which the area of said conductive zone (24) is at least 400 cm².

4: The therapeutic knitted article of claim 1, in which the knit and the yarns of said article are selected in such a manner as to form a compressive orthosis suitable for delivering therapeutic compressive support that is degressive from the ankle, with support pressure at the ankle of at least 10 mmHg (13.3 hPa).

5: The therapeutic knitted article of claim 1, including an electrically conductive top region (26) separated from said conductive zone (24) by a non-conductive intermediate region (28), said intermediate region (28) having an electrical connection (32) crossing it to connect the top region (26) to the conductive zone (24) so that the top region forms a connection strip or area for a wire for making a connection to a terminal of an electrostimulation generator.

6: The therapeutic knitted article of claim 5, in which said electrical connection (32) is a narrow knitted strip of the article including a knit yarn and/or a filler yarn that is electrically conductive.

7: The therapeutic knitted article of claim 1, in which the conductive yarn (18) is a yarn including a thread (22) of conductive material.

8: The therapeutic knitted article of claim 7, in which said conductive material is a material from the group comprising silver, gold, copper, and copper oxide.

9: The therapeutic knitted article of claim 7, in which the thread of conductive material is a covering thread.

10: The therapeutic knitted article of claim 9, in which the conductive thread is a parallel covering thread.

11: The therapeutic knitted article of claim 1, in which the face of the conductive zone that is to come into contact with the skin of the lower limbs is that one of its faces which presents the largest surface area of conductive yarn.

12: A therapeutic appliance for treating functional disorders of venous insufficiency in the lower limbs and for preventing deep vein thrombosis, the appliance comprising in combination:
an electromyostimulation generator (12) suitable for delivering pulses of current between its output terminals;
a device (10) according to claim 1; and
a connection wire (14) electrically connecting the electrically conductive region of said device to a respective terminal of the electromyostimulation generator.
